SEQUENCE LISTING (15 sequences)

SEQ ID NO: 1 - protein, 22 aa, Artificial Sequence
  synthetic amino acid sequence
LATNSELVGTLTRSCKDETFLL

SEQ ID NO: 2 - protein, 25 aa, Artificial Sequence
  synthetic amino acid sequence
MGLGKTIQTIALITYLMEHKRINGP

SEQ ID NO: 3 - protein, 30 aa, Artificial Sequence
  synthetic amino acid sequence
SRWTEEEMEVAKKGLVEHGRNWAAIAKMVG

SEQ ID NO: 4 - protein, 25 aa, Artificial Sequence
  synthetic amino acid sequence
GQLNSDHKSQLVEKVRLKIGCGIQL

SEQ ID NO: 5 - protein, 33 aa, Artificial Sequence
  synthetic amino acid sequence
NRAGFEASDPRIIRLISLAAQKFISDIANDALQ

SEQ ID NO: 6 - protein, 6 aa, Artificial Sequence
  synthetic amino acid sequence
PKKRKV

SEQ ID NO: 7 - protein, 6 aa, Artificial Sequence
  synthetic amino acid sequence
ARRRRP

SEQ ID NO: 8 - protein, 10 aa, Artificial Sequence
  synthetic amino acid sequence
EEVQRKRQKL

SEQ ID NO: 9 - protein, 9 aa, Artificial Sequence
  synthetic amino acid sequence
EEKRKRTYE

SEQ ID NO: 10 - protein, 16 aa, Artificial Sequence
  synthetic amino acid sequence
KRPAATKKAGQAKKKK

SEQ ID NO: 11 - protein, 32 aa, Artificial Sequence
  synethetic amino acid sequence
RPKKRKVRRRLATNSELVGTLTRSCKDETFLL

SEQ ID NO: 12 - protein, 35 aa, Artificial Sequence
  synthetic amino acid sequence
RPKKRKVRRRMGLGKTIQTIALITYLMEHKRINGP

SEQ ID NO: 13 - protein, 40 aa, Artificial Sequence
  synthetic amino acid sequence
RPKKRKVRRRSRWTEEEMEVAKKGLVEHGRNWAAIAKMVG

SEQ ID NO: 14 - protein, 35 aa, Artificial Sequence
  synthetic amino acid sequence
RPKKRKVRRRGQLNSDHKSQLVEKVRLKIGCGIQL

SEQ ID NO: 15 - protein, 43 aa, Artificial Sequence
  synthetic amino acid sequence
RPKKRKVRRRNRAGFEASDPRIIRLISLAAQKFISDIANDALQ

(19) United States
(12) Patent Application Publication
(10) Pub. No.: US 2008/0306001 A1
(43) Pub. Date: Dec. 11, 2008

(54) TRANSCRIPTIONAL MODULATION OF EXTRACELLULAR MATRIX (ECM) OF DERMAL FIBROBLASTS

(76) Inventors: Anzelika Liik, Tallinn (EE); Ave Laas, Tallinn (EE); Rita Zobel, Tallinn (EE); Toomas Neuman, Tallinn (EE)

Correspondence Address: BIOTECH BEACH LAW GROUP , PC, 625 BROADWAY, Suite 1210, SAN DIEGO, CA, 92101, US

(21) Appl. No.: 12/062,155

(22) Filed: Apr. 3, 2008

Related U.S. Application Data
(60) Provisional application No. 60/921,712, filed on Apr. 4, 2007.

(51) Int. Cl. A61K 38/00; C07K 14/00; C12N 5/06; A61P 17/00; C12N 9/50

(52) U.S. Cl. 514/12; 530/324; 435/219; 530/356; 435/375

(57) ABSTRACT

The present invention includes peptides, compositions as well as their use for the prevention or treatment of various age related or pathological conditions of skin or other tissues including skin wrinkles, wounds, different types of fibrosis and methods of reconstructing different tissues such as techniques used in regenerative medicine. The invention further includes peptide mimetics and methods of use including interfering with transcriptional complexes characteristic of fibroblasts of aged skin and stimulating synthesis of structural components of extracellular matrix.

### CROSS REFERENCE TO RELATED APPLICATIONS

The present application claims benefit of priority to U.S. patent application Ser. No. 60/921,712 filed on Apr. 4, 2007 which is herein incorporated by reference in its entirety.

### TECHNICAL FIELD

The present invention relates to peptides, compositions and their use for the prevention or treatment of various age related or pathological conditions of skin or other tissues such as but not limited to skin wrinkles, wounds, different types of fibrosis and methods of reconstructing different tissues such as techniques used in regenerative medicine. More specifically the present invention includes compositions including peptide mimetics and methods of use including interfering with transcriptional complexes characteristic of fibroblasts of aged skin and stimulating synthesis of structural components of extracellular matrix.

### BACKGROUND OF THE INVENTION

Wrinkles and the appearance or characteristics of “old” skin can have a profound impact on self-esteem. Indeed, the stigma attached to looking old is evidenced by the fact that Americans spend more than $12 billion each year on cosmetics to camouflage the signs of aging.

As a person ages, the skin undergoes significant changes. Among these include a decreased rate of cell division. Decreasing cell division within the dermal region of the skin results in a thinning of the inner layer of skin. Because the dermis in part functions as a structural layer adding to the overall strength of the skin, thinning of the dermis results in a skin that is more vulnerable to injuries and damage. Since the ability of the skin to repair itself also diminishes with age, wounds are slower to heal. Similarly, skin damage has also been found to accelerate upon prolonged exposure to ultra-violet (UV) radiation.

The extracellular matrix (ECM) includes an underlying network of elastin and collagen fibers and provides scaffolding for the surface skin layers. It also contains proteoglycans, numerous fibrillar proteins (fibrillins, fibulins), metalloproteinases, enzymes such as lysyl oxidase and many other minor components. When damaged, the ECM loosens and unravels causing skin to lose its elasticity. The damage of the ECM may be caused by damage to the collagen fibers, the major structural protein in the skin, by the accumulation of abnormal elastin, the protein that allows tissue to stretch, or by abnormal activity of metalloproteinases, proteins that repair damaged skin. Abnormal elastin accumulation correlates to increased amounts of enzymes called metalloproteinases. (One study indicated that when people with light to moderate skin color are exposed to sunlight for just five to 15 minutes, metalloproteinases remain elevated for about a week.) The normal function of metalloproteinases is generally positive; they remodel the damaged tissue by reforming collagen. This is an imperfect process, however, and some of metalloproteinases produced by damaging conditions degrade collagen. The result is an uneven formation (matrix) of disorganized collagen fibers. Repetition of this imperfect skin rebuilding over and over again is believed to cause or influence the appearance of wrinkles.

Collagen is a structural protein found in the ECM. Collagens and proteins with collagen-like domains form large superfamilies, and the numbers of known family members are increasing constantly. Vertebrates have at least 27 collagen types with 42 distinct polypeptide chains, >20 additional proteins with collagen-like domains and 20 isoenzymes of various collagen-modifying enzymes (Myllyharju and Kivirikko. 2004. Collagens, modifying enzymes and their mutations in humans, flies and worms. Trends in Genetics Vol. 20, pp. 33-43). Collagens are the most abundant proteins in the human body, constituting 30% of its protein mass. The important roles of these proteins have been clearly demonstrated by the wide spectrum of diseases caused by a large number of mutations found in collagen genes. More than 1300 mutations have so far been characterized in 23 of the 42 human collagen genes in various diseases. All collagen molecules consist of three polypeptide chains, called a chains, and contain at least one domain composed of repeating Gly-X-Y sequences in each of the constituent chains. In some collagens all three a chains are identical, whereas in others the molecules contain two or even three different a chains. Skin fibroblasts synthesize different types of collagen. The most abundant collagen molecules in dermal ECM are collagen I, collagen II, collagen IV and collagen V.

The level of individual collagen molecules in ECM is controlled by synthesis and degradation. Synthesis of different collagen molecules is largely controlled by transcriptional mechanisms. Transcription factors SMAD, SP, ETS STAT and several others mediate the effect of variety of signaling systems at the transcriptional level. Cytokines, interleukins and TGFbeta are the signaling molecules that control homeostasis of dermal ECM and mediate effect of immune and humoral systems.

Elastin fibers function together with collagen in the ECM and provide elasticity to the system. Elastic fibers are essential extracellular matrix macromolecules comprising an elastin core surrounded by a mantle of fibrillin-rich microfibrils (Kielty, Sherratt and Shuttleworth. 2002. Elastic fibers. Journal of Cell Science 115, 2817-2828). They endow connective tissues such as blood vessels, lungs and skin with the critical properties of elasticity and resilience. The biology of elastic fibers is complex because they have multiple components, a tightly regulated developmental deposition, a multi-step hierarchical assembly and unique biomechanical functions. Elastic fibres are designed to maintain elastic function for a lifetime. However, various enzymes (matrix metalloproteinases and serine proteases) are able to cleave elastic fiber molecules (Kielty et al., 1994; Ashworth et al., 1999c). Indeed, loss of elasticity due to degradative changes is a major contributing factor in ageing of connective tissues, in the development of aortic aneurysms and lung emphysema, and in degenerative changes in sun-damaged skin (Watson et al., 1999). The importance of elastic fibres is further highlighted by the severe heritable connective tissue diseases caused by mutations in components of elastic fibers (for reviews, see Robinson and Godfrey, 2000; Milewicz et al., 2000). Functional elastic fibers contain more that 30 different molecules that all possess specific functions.

Elastin gene expression is regulated by several extracellular effectors such as IL-1b, bFGF, IGF-1, TNF-alpha and TGF-beta that initiate complex intracellular responses. At the transcriptional level these pathways modify activity of NF-kB, C/EBP. FRA, SMAD, SP and AP-1 transcription factors.

Transcriptional regulators determine regulatory networks that control gene-specific transcription. The misregulation of these networks is correlated with a growing number of human diseases that are characterized by altered gene expression patterns. This has spurred intense efforts toward the development of artificial transcriptional regulators and/or molecules that modify activity of transcriptional regulators to correct and restore “normal” expression of affected genes. Numerous research groups are focusing on development of treatment strategies that target signaling systems, mostly kinases and phosphatases, and cell surface molecules that control gene expression and regulate cell division and differentiation. All potential treatments that target signaling and cell surface molecules have one critical problem—cell type specificity. To be effective with minimal side effects, treatments have to affect only diseased cells. Signaling systems and surface molecules are expressed and function in a wide variety of cell populations that makes achieving localized/restricted effects extremely difficult.

The present invention addresses the need for compositions and methods for the treatment of various skin conditions associated with aging or skin damage. The present invention exploits the specific composition of transcriptional complexes characteristic for fibroblasts of the aged skin to stimulate synthesis of fibrillar components of the ECM using mimicking peptides that suppress activity of inhibitory transcriptional modulators characteristic for old skin.

### SUMMARY

The present invention includes peptides, compositions and methods for the treatment of dermal fibroblasts to stimulate synthesis of components of ECM. The peptides and compositions include peptide mimetics designed to modulate the activity of particular transcription complexes by interfering with the functioning of transcriptional regulators that inhibit transcription.

In one aspect of the present invention a composition is provided including a peptide mimetic useful for the treatment of dermal fibroblasts. The peptide mimetics provided herein correspond to functional domains of particular transcription regulators, which the present invention establishes are involved in the synthesis of ECM components. In a preferred embodiment, a peptide mimetic corresponds to the transrepression domain of a transcription regulator present in a dermal fibroblast. In another preferred embodiment, a peptide mimetic corresponds to the multimerization domain of a transcription regulator present in a dermal fibroblast. In another preferred embodiment, a peptide mimetic corresponds to the protein:protein interaction domain of a transcription regulator present in a dermal fibroblast. The disclosed peptide mimetics are capable of stimulating synthesis of fibrillary components of dermal ECM. Exemplary peptides capable of stimulating synthesis of components of ECM, such as from human fibroblasts include those provided in SEQ ID NOS: 1-5 and 11-16.

In another aspect of the present invention a pharmaceutical is provided including a peptide mimetic of the present invention and a pharmaceutically or physiologically acceptable carrier.

In yet another aspect of the present invention a method of treating or preventing a skin condition associated with aging is provided including providing an individual having a skin condition associated with aging and administering a peptide, composition, pharmaceutical composition or cosmetic formulation of the present invention.

In still another aspect of the present invention, a method of synthesizing a structural component of a extracellular matrix (ECM) is provided including administering a peptide, composition or pharmaceutical of the present invention to an ECM, the dermis of an individual or a fibroblast.

### DETAILED DESCRIPTION

### A. Definitions

Unless defined otherwise, all technical and scientific terms used herein have the same meaning as is commonly understood by one of ordinary skill in the art to which this invention belongs. All publications referred to herein are incorporated by reference in their entirety. In the event that there are a plurality of definitions for a term provided herein, those in this section prevail unless stated otherwise.

The term “correspond” as used herein refers to direct correspondence or substantial similarity. Peptide mimetics substantially similar to fragments of transcription regulators preferably have about 80%, more preferably about 85%, more preferably about 90%, more preferably about 95% identity to the amino acid sequence of the fragment. Such peptide mimetics are capable of competing with such fragments of transcription regulator for binding to biological binding partners of the transcription regulators.

The term “linked” as used herein refers to the association of two or more elements or portions of the compositions to one another. For example, peptide mimics of the present invention may be linked to a cell penetrating peptide (CPP) or an intracellular targeting signal. A peptide or composition of the present invention may include one or more linked elements or portions.

The term “old skin” or “aged skin” includes skin such as human skin that demonstrates symptoms of aging. Such symptoms may include wrinkles, decreased elasticity, thinning or increased susceptibility to cutting or tearing. Additional symptoms may include a slow response for wound healing from trauma such as a cut or incision.

The term “modulation” or “modulating” as used herein refers to an increase or decrease in activity. The present invention may modulate a particular or desired transcription regulator.

The term “multimerization domain” of a transcription regulator as used herein refers to the protein domain of a transcription regulator that is involved in homo- or heteromultimerization, including dimerization. For example, the helix-loop-helix domain of the bHLH factor ITF2 is involved in homo- and heterodimerization.

The term “peptide” as used herein refers to at least two covalently attached amino acids. The peptide may be composed of naturally occurring and synthetic amino acids, including amino acids of (R) or (S) stereo configuration. The term “peptide” is not intended to include an upper limit of size or number of amino acid residues. A polypeptide is intended to be encompassed within the term “peptide.”

The term “pharmaceutically acceptable” as used herein refers to approved or approvable by a regulatory agency of the Federal or a state government for use in animals, and more particularly in humans. The term “pharmaceutically acceptable carrier” refers to an approved or approvable diluent, adjuvant, excipient or carrier with which a peptide or composition is administered.

The term “prodrug” as used herein refers to a composition that, upon in vivo administration, is metabolized by one or more steps or processes or otherwise converted to the biologically, pharmaceutically or therapeutically active form of the peptide or composition. To produce a prodrug, the pharmaceutically active peptide or composition is modified such that it will be regenerated by metabolic processes. The prodrug may be designed to alter the metabolic stability or the transport characteristics of a drug, to mask side effects or toxicity, to improve the flavor of a drug or to alter other characteristics or properties of a drug.

The term “protein:protein interaction domain” of a transcription regulator as used herein refers to the domain of a transcription regulator that is involved in protein:protein interactions other than those with the basal transcription apparatus or multimerization partners. Examples of protein:protein interaction domains are those possessing nuclear localization signals, those that bind to chaperone proteins, and those that bind to corepressors or coactivators.

The term “purified” or “isolated” as used herein refers to a peptide or composition that is substantially free from the environment in which it is synthesized or expressed, and in a form where it can be practically used. In some embodiments, purified or isolated is a substantially pure composition, i.e., more than 90% pure, preferably more than 95% pure, and preferably more than 99% pure.

The term “therapeutically effective amount” refers to the amount of a peptide or composition that, when administered to a patient for treating a disease or disorder, is sufficient to affect such treatment for the disease or disorder. A “therapeutically effective amount” may reduce a symptom associated with the disorder or condition. The “therapeutically effective amount” may vary depending on the peptide or composition, the skin condition and its severity and the age and weight of the patient to be treated.

The term “transactivation and transrepression domain” of a transcription regulator as used herein refers to the protein domain of a transcription regulator that interacts with the basal transcription complex components, i.e., one or more components of the transcriptional preinitiation complex, including RNA Pol II.

The term “transcription regulator” as used herein refers to any component of transcriptional complex, DNA binding or non-DNA binding.

As used herein, the abbreviations for any protective groups, amino acids and other compositions, are, unless indicated otherwise, in accord with their common usage, recognized abbreviations, or the IUPAC-IUB Commission on Biochemical Nomenclature (see, Biochem. 1972 11:942-944).

### B. Introduction of the Invention

The present invention includes peptides, compositions, pharmaceuticals, cosmetic formulations and methods for the prevention or treatment of a variety of skin conditions. The treatment methods include but are not limited to the stimulation of ECM synthesis in dermal fibroblasts.

Dermal cells, mostly fibroblasts keep skin young by generating novel cells that synthesize and deposit new ECM during differentiation process. Aging is related to reduction of skin stem cells and differentiation of these cells. One approach to fight with aging is to stimulate renewal of dermal cells. Alternative is to stimulate ECM production and homeostasis in dermis.

The present invention is related to stimulation of ECM production. Significant differences have been identified in the transcriptional machinery of fibroblasts isolated from young and old skin. Transcription of specific genes is controlled by DNA binding transcription factors (TF) and a complex of transcriptional regulators that do not bind DNA but control activity of RNA polymerase II. These regulators form complexes with specific functions such as general transcription factors (GTF), TATA binding protein associated factors (TAFs), chromatin remodeling factors (SWI/SNF complex) and mediator complex (MED). The present invention demonstrates that complexes of transcriptional regulators in old and young skin fibroblasts have different composition that affects transcription of ECM components. Old skin fibroblasts express regulators that inhibit transcription of ECM components. Mimicking peptides have been developed that interfere with the function of these inhibitory transcriptional regulators and therefore stimulate synthesis of ECM in old fibroblasts. These peptides can be used to remodel ECM of old fibroblasts and rejuvenate old skin that makes them favorable cosmetic products. Also, these peptides can be used to stimulate wound healing and treat other conditions related to dysfunction of fibroblasts.

The peptide mimetics of the invention are capable of modulating the activity of their corresponding transcription regulator in dermal fibroblasts. Precise temporal and spatial regulation of the transcription of protein-encoding genes by RNA polymerase II (pol II) is important to the execution of complex gene expression programs in response to growth, developmental and homeostatic signals. The molecular circuitry that enables coordinated gene expression is largely based on DNA-binding transcription factors (TFs) that bring regulatory information to the target genes. As a rule, DNA binding TFs do not interact directly with polII and other basal transcriptional complex components. Groups of factors called coactivators and Mediator complex have emerged as central players in the process by which TF binding information is converted into the appropriate response by pol II. Although it has been realized that coactivators and Mediator complex are universally required for the expression of almost all genes, the full implications of a requirement for a multi-subunit coactivator complex are not yet readily apparent. By inserting itself between the DNA binding TFs and the basal transcriptional machinery, the Mediator complex probably affords additional opportunities to control the diverse regulatory inputs received both from the DNA-binding factors and, most likely, from other signals and to present an appropriately calibrated output to the pol II machinery. In its capacity as a processor of diverse signals in the form of activators, repressors, and coactivators that impinge on it, and its location at the interface of pol II and general transcription factors (GTFs), the Mediator represents a final check-point before pol II transcription actually commences. Its central role in transcriptional control makes Mediator complex an attractive drug target that enables researchers and clinicians to control and correct expression of a large number of genes. Mediator complex contains up to 30 subunits whereas its composition in different cell types and on different promoters varies and contains different members of Mediator family of coactivators. This cell specific variability of Mediator complex assures specificity of potential treatments that target Mediator complex.

It is well known that transcriptional control of individual genes is cell type specific and that different transcription factor complexes are responsible for this specificity. The present invention includes the use of a cell type specificity of TF complexes to control expression of proteins that are critical for ECM functioning. Achieving this goal allows the manipulation of synthesis of specific components of ECM. For a long time TFs have been considered as difficult targets for effective drug development. Recently numerous reports show that small molecules can be developed that interact with specific domains of TFs and control activity of specific TF complexes. Our experimental work shows that function of TFs can be modified (stimulated, repressed) using mimicking/interfering peptides that are targeted using a combination of cell penetrating peptides (CPP) and nuclear localization signal (NLS) sequences.

The ultimate action of TFs on target genes, after site-specific DNA binding, is to enhance the recruitment and/or function of the general transcription machinery (RNA polymerase II and general transcription factors TFII-A, -B, -D, -E, —F, and —H; Roeder, 1996) on cognate core promoter elements. Recent studies have implicated a large multisubunit coactivator complex, a Mediator, as the main pathway for direct communication between DNA binding TFs and the general transcription machinery (reviewed in Malik and Roeder, 2000). Large number of protein/protein interactions determines specificity and function of Mediator complex. Peptides that mimic interaction surfaces of different transcription factors have been designed and used to manipulate expression of target genes (Kalinichenko et al., 2004, Chinmay et al., 2005, Gail et al., 2005).

A critical issue in development of mimicking peptides is modeling TF complexes and identification of interacting protein surfaces. Prediction of the structures of multimolecular complexes has largely not been addressed, probably due to the magnitude of the combinatorial complexity of the problem. Docking applications have traditionally been used to predict pair wise interactions between molecules. Several algorithms that extend the application of docking to multimolecular assemblies have been developed. We apply these algorithms to predict quaternary structures of both oligomers (few interacting proteins) and multi-protein complexes. These algorithms have predicted well a near-native arrangement of the subunits of Mediator complexes.

Another critical issue is delivery of therapeutic peptides to cell nucleus where transcription factor complexes are localized and where they perform their function. Cell membranes act as protective walls to exclude peptides that are not actively imported by living cells. In order to overcome this barrier for effective delivery of membrane-impermeable peptides, several chemical and physical methods have been developed including electroporation and cationic lipids/liposomes. These methods have been shown to be effective for delivering variety of macromolecules. The drawbacks of these harsh methods are, primarily, the unwanted cellular effects exerted by them, and, secondly, their limitation to in vivo applications. The last decade's discovery of cell-penetrating peptides (CPP) translocating themselves across cell membranes along with a cargo 100-fold their own size, via a seemingly energy independent process, opens up the possibility for efficient delivery of proteins, peptides and small molecules into cells both in vitro and in vivo. The only consistently found feature present in all CPPs is the high content of basic amino acids, resulting in a positive net charge. Rothbard et al. (2000) showed that cyclosporin A was efficiently delivered into dermal T lymphocytes and inhibited inflammation by linking to a hepta-arginine segment, suggesting that positive charge is the required feature for cellular translocation. CPPs possess an appealing set of desirable features for cellular targeting, such as effective delivery in vivo, targeting of the nucleus, applicability to all cell types, no apparent size constraint of cargo and seemingly no immunogenic, antigenic or inflammatory properties. As delivery vectors, cell-penetrating peptides definitely have proven their value. Their ability to effectively deliver hydrophobic macromolecules into practically all types of cells in vitro, as well as in vivo, without marked levels of cytotoxicity, is impressive. The present invention includes combining CPPs with mimicking peptides to target transcriptional complexes.

### C. Peptides and Compositions

The present invention includes a variety of peptide mimetics and related compositions, nucleic acid molecules encoding the peptide mimetics and methods of their use to stimulate ECM synthesis in dermal fibroblasts. The peptide mimetics provided correspond to fragments of inhibitory transcription regulators present in dermal fibroblasts. The complicated nature of transcription factor activity, the multiple interactions that transcription factors exhibit to modulate transcription of a particular gene, and the genetic variation between individuals makes it difficult to predict whether administration of a particular peptide will have a beneficial effect on a particular individual.

The present invention provides peptide mimetics that are efficacious in the treatment of dermal fibroblasts to stimulate synthesis of ECM. Moreover, the present invention establishes that the transcription regulators to which the exemplified peptide mimetics correspond play a role in controlling ECM homeostasis in skin and other fibroblasts. Accordingly, the present invention includes peptide mimetics which mimic particular functional domains of the identified transcription regulators and are capable of modulating their activity. The efficacy in dermal fibroblast treatment of any particular peptide mimetic directed to any one of the identified transcription regulators is easily tested by routine experimentation using, for example, the in vitro and in vivo assays described herein.

A transcription regulator functional domain interacts with a binding partner for effect. By providing peptide mimetics having efficacy in the treatment of dermal fibroblasts, the present invention also identifies binding partners involved in synthesis of ECM. These binding partners are proteins that are components of transcriptional complexes. Peptides corresponding to the counterpart binding domains of binding partners which interact with functional domains of transcription regulator are also provided herein as peptide mimetics useful for the treatment of dermal fibroblasts.

In some embodiments peptides or compositions of the present invention include peptide mimetics having about 40 amino acids in length or less, more preferably about 35 amino acids or less, more preferably about 30 amino acids or less, more preferably about 25 amino acids in length, though shorter peptides may be used. The peptide mimetic may be included in a larger peptide, which may include heterologous sequences at one end or both ends of the peptide. The peptide may have at one end, one or more signal sequences such as a nuclear localization signal (NLS) sequence or a cell penetrating peptide (CPP). Sequences or chemical groups may be added to increase peptide stability or otherwise alter the peptide mimetics properties in desirable ways.

Peptides including non-naturally occurring amino acids may be synthesized or, in some cases, made by recombinant techniques (van Hest, J. C. et al., FEBS Lett. 428: 68-70 (1998); and Tang et al., Abstr. Pap. Am. Chem. S218: U138-U138 Part 2 (1999)), both of which are expressly incorporated by reference herein in their entirety. Isolated nucleic acid molecules encoding such peptides, including but limited to those that encode the amino acid sequences provided in SEQ ID NOS: 1-15, are also encompassed within the present invention and are intended to enompass variations in nucleic acid sequence with respect to the degeneracy of the genetic code. Peptides may be generated wholly or partly by chemical synthesis. Peptides and compositions of the present invention can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Peptide mimetics may include modified amino acids (e.g., phosphorylated, ubiquitinated, acetylated, sulfated, methylated, etc.) designed to resemble transcription factor functional domains following posttranslational modification. Many post-translational modifications, including phosphorylation, are known to regulate the activity and interactions of transcription factors. In some embodiments the amide nitrogen of an asparagine amino acid side chain is glycosylated (N-lined glycosylation). In other embodiments, the hydroxyl oxygen of a serine or threonine amino acid side chain is glycosylated (O-linked glycosylation). Peptide mimetics that resemble the modified form of functional domains, as well as those that resemble the unmodified form of functional domains are included.

Preferred peptide mimetics for use in the methods of stimulating synthesis of ECM components correspond to functional domains of particular transcription regulators present in dermal fibroblasts.

Preferred peptide mimetics include:

LATNSELVGTLTRSCKDETFLL (SEQ ID NO: 1) directed to TATA binding protein associated factor TAF4;

MGLGKTIQTIALITYLMEHKRINGP (SEQ ID NO: 2) directed to SWI/SNF chromatin remodeling complex member Brg1/SMARCA4;

SRWTEEEMEVAKKGLVEHGRNWAAIAKMVG (SEQ ID NO: 3) directed to nuclear hormone co-repressor N-CoR1;

GQLNSDHKSQLVEKVRLKIGCGIQL (SEQ ID NO: 4) directed to SMAD7 transcription factor that functions as a negative regulator of TGFbeta signaling; and

NRAGFEASDPRIIRLISLAAQKFISDIANDALQ (SEQ ID NO: 5) directed to TAF family transcription factor TAF10.

The present invention also includes functional variants of the disclosed peptide mimetics, which can be made using techniques known in the chemical or biological arts. A functional variant or functional peptide refers to a peptide which possesses the biological function or activity identified through a defined functional assay, and which is associated with a particular biologic activity (i.e., stimulation of synthesis of ECM components). In one embodiment, the variants are substitutional changes of one or more residues to a native functional domain sequence.

In some embodiments, the peptide mimetics are conservative variants of the exemplary mimetic sequences disclosed above. Conservative variants as used herein refer to the replacement of an amino acid by another chemically and biologically similar residue. Examples of conservative variations include the substitution of one hydrophobic residue such as isoleucine, valine, leucine, or methionine for another; the substitution of one polar residue for another polar residue, such as substitution of one arginine for lysine, glutamic acid for aspartic acid, or glutamine for asparagine; and the substitution of one hydroxylated amino acid serine or threonine for another.

In other embodiments, the changes are deletions or insertions of a few residues, more preferably one residue. Changes should preserve at least a portion of biological activity, however an increase or decrease in biological activity may result. Preferably the changes do not substantially decrease biological activity of the peptide. Amino acids may be added to the amino or carboxy terminus. Biological activity is readily tested by synthesizing the substitution, insertion, or deletion variants of the peptide mimetics and assaying for efficacy using the methods described and exemplified herein.

As nonlimiting examples, the terminal amino group or carboxyl group of the peptide mimetic may be modified by alkylation, amidation, or acylation to provide esters, amides or substituted amino groups, where the alkyl or acyl group may be of from about 1 to 30, usually 1 to 24, preferably either 1 to 3 or 8 to 24, particularly 12 to 18, carbon atoms. The peptide or derivatives thereof may also be modified by acetylation or methylation to alter the chemical properties, for example lipophilicity. Other modifications include deamination of glutamyl and asparaginyl residues to the corresponding glutamyl and aspartyl residues, respectively; hydroxylation of proline and lysine; phosphorylation of hydroxyl groups of serine or threonine; and methylation of amino groups of lysine, arginine, and histidine side chains (see, e.g., Creighton, T. E., Proteins: Structure and Molecular Properties, W.H. Freeman & Co. San Francisco, Calif. (1983)).

As additional nonlimiting embodiments, the peptides of the present invention may include one or more peptides having one or more of the following modifications: peptides wherein one or more of the peptidyl —C(O)NR— linkages (bonds) have been replaced by a non-peptidyl linkage such as a —CH2-carbamate linkage (—CH2OC(O)NR—), a phosphonate linkage, a —CH2-sulfonamide (—CH2-S(O)2NR—) linkage, a urea (NHC(O)NH—) linkage, a —CH2-secondary amine linkage, or with an alkylated peptidyl linkage (—C(O)NR—) wherein R is C1-C4 alkyl; peptides wherein the N-terminus is derivatized to a —NRC(O)R group, to a —NRC(O)OR group, to a NRS(O)2R group, to a —NHC(O)NHR group where R and R± are hydrogen or alkyl with the proviso that R and R1 are not both hydrogen; peptides wherein the C terminus is derivatized to —C(O)R2 where R2 is selected from the group consisting of C1-C4 alkoxy, and —NR3R4 where R3 and R4 are independently selected from the group consisting of hydrogen and C1-C4 alkyl.

The peptide mimetic compositions of the invention may comprise a translocation agent, preferably a cell penetrating peptide, which facilitates translocation of an associated peptide mimetic across a cell membrane. It is known that certain peptides have the ability to penetrate a lipid bilayer (e.g., cell membranes) and translocate an attached cargo across the cell membrane. This is referred to herein as “translocation activity”. Without being bound by theory, these membrane penetrating peptides appear to enter the cell, in part, via non-endocytic mechanisms, as indicated by the ability of the cell penetrating peptides to enter the cell at low temperatures (e.g., 4° C.) that would normally inhibit endocytic, receptor-based, internalization pathways. Peptides with cell penetrating properties include, by way of example and not limitation, penetratins, Tat-derived peptides, signal sequences (i.e., membrane translocating sequences), arginine-rich peptides, transportans, amphipathic peptide carriers, and the like (see, e.g., Morris, M. C. et al., Nature Biotechnol. 19:1173-1176 (2001); Dupont, A. J. and Prochiantz, A., CRC Handbook on Cell Penetrating Peptides, Langel, Editor, CRC Press, (2002); Chaloin, L. et al., Biochemistry 36(37):11179-87 (1997); and Lundberg, P. and Langel, U., J. Mol. Recognit. 16(5):227-233 (2003); all publications incorporated herein by reference).

It is to be understood that the cell penetrating peptides may include naturally occurring amino acids or contain at least one or more D-amino acids and amino acid analogues. In another embodiment, the cell penetrating peptides may comprise all D amino acids. As used herein, the term “amino acid” is applicable not only to cell membrane-permeant peptides, but also to peptide inhibitors of cell penetrating peptides, any linker moieties, subcellular localization sequences, and peptide cargos, including peptide pharmaceutical agents (i.e., all the individual components of the present compositions).

The incorporation of non-natural amino acids, including synthetic non-native amino acids, substituted amino acids, or one or more D-amino acids into the peptides (or other components of the composition, with exception for protease recognition sequences) may be desirable in certain situations. D-amino acid-containing peptides exhibit increased stability in vitro or in vivo compared to L-amino acid-containing forms. Thus, the construction of peptides incorporating D-amino acids may be particularly useful when greater in vivo or intracellular stability is desired or required. More specifically, D-peptides are resistant to endogenous peptidases and proteases, thereby providing better oral transepithelial and transdermal delivery of linked drugs and conjugates, improved bioavailability of membrane-permeant complexes, and prolonged intravascular and interstitial lifetimes when such properties are desirable. The use of D-isomer peptides can also enhance transdermal and oral transepithelial delivery of linked drugs and other cargo molecules. Additionally, D-peptides cannot be processed efficiently for major histocompatibility complex class II-restricted presentation to T helper cells, and are therefore less likely to induce humoral immune responses in the whole organism. Peptide conjugates can therefore be constructed using, for example, D-isomer forms of peptide membrane permeant sequences, L-isomer forms of cleavage sites, and D-isomer forms of bioactive peptides.

To increase specificity, the compositions provided herein may further comprise an intracellular targeting, or subcellular localization signal, to target the peptide mimetics to a specific subcellular compartment, particularly the nucleus. In embodiments wherein the compositions comprise both a cell penetrating peptide and an intracellular targeting signal, the intracellular targeting signal may be separate from a cell penetrating peptide, may overlap with a cell penetrating peptide, or may be subsumed by a cell penetrating peptide of the composition. Thus, in some embodiments, translocation and intracellular targeting activities are conferred by partially or completely overlapping regions of the composition, while in other embodiments these activities are conferred by separate segments. In a particularly preferred embodiment, a nuclear localization signal is merged with a cell penetrating peptide in a composition.

In some preferred embodiments, the compositions include a subcellular targeting sequence such as a nuclear localization sequence (NLS). Generally, nuclear localization sequences are characterized by a short single cluster of basic amino acids (monopartite) or two clusters of basic amino acids separated by a 10-12 amino acid linking region (bipartite structure) and functions to direct the entire protein in which they occur to the cell's nucleus. NLS amino acid sequences used in the art include those from SV40 large T Antigen, with the sequence PKKRKV (Kalderon et al., Cell 39:499-509 (1984)) (SEQ ID NO: 6); the human retinoic acid receptor β-nuclear localization signal sequence ARRRRP (SEQ ID NO: 7); the NFκB p50 associated sequence EEVQRKRQKL (Ghosh et al., Cell 62:1019 (1990)) (SEQ ID NO: 8); and NFκB p65 associated sequence EEKRKRTYE (Nolan et al., Cell 64:961 (1991)) (SEQ ID NO: 9). Bipartite nuclear localization activity is described in Boulikas, J. Cell. Biochem. 55(1):32-58 (1994), Dingwall, et al., J. Cell Biol. 107:641-849 (1988) (e.g., double basic NLS's exemplified by nucleoplasmin associated sequence KRPAATKKAGQAKKKK (SEQ ID NO: 10)), Kalderon, D. et al., Cell 39:499-509 (1984), and Robbins, J. et al., Cell 64:615-623 (1991). NLS amino acid sequences may be combined or linked using techniques provided throughout the present invention with the peptide mimetics, including but not limited to those provided in SEQ ID NOS: 1-5. All publications are herein incorporated by reference in their entirety.

Non-peptide covalent bonds may also be used to link the various elements to the peptide mimetic in the compositions. Chemical ligation methods may be employed to create a covalent bond between elements in the compositions if desired. Electrostatic interactions may also be used to join negatively-charged elements and positively-charged elements in the compositions. Combinations of linkage schemes may also be used.

The peptides and compositions can be purified by art-known techniques such as but not limited to reverse phase chromatography, high performance reverse chromatography, ion exchange chromatography, gel electrophoresis, affinity chromatography, molecular sieve chromatography, isoelectric focusing, and the like. In a preferred embodiment, the peptides and compositions of the present invention may be purified or isolated after synthesis or expression. The peptides and compositions may also be purified by selective solubility, for instance in the presence of salts or organic solvents. The degree of purification necessary will vary depending on use of the subject compositions. Thus, in some instances no purification will be necessary. In some instances the peptide or composition is greater than 99% pure. In other embodiments the peptide or composition is greater than 95% pure. In other embodiments the peptide or composition is greater than 90% pure.

The compositions of the present invention include one or more of the peptides, as well as their salts and their prodrugs, if applicable. The salts or prodrugs should retain a portion of the desired biological activity of the parent peptide or composition or be provided in a form that the body or subject can convert to a biologically active form. The salts, for example, can be formed between a positively charged substituent (e.g., amino) on a peptide or composition and an anion. Suitable anions include, but are not limited to, chloride, bromide, iodide, sulfate, nitrate, phosphate, citrate, methanesulfonate, tartrate, trifluoroacetate, and acetate. Likewise, a negatively charged substituent (e.g., carboxylate) on a peptide or composition can form a salt with a cation. Suitable cations include, but are not limited to, sodium ion, potassium ion, magnesium ion, calcium ion, and an ammonium cation such as teteramethylammonium ion. Examples of prodrugs include esters and other pharmaceutically acceptable derivatives, which, upon administration to a subject, are capable of providing the peptides or compositions described above.

The peptides and compositions of the present invention may be formulated for administration for the prevention or treatment of a variety of medical conditions. Pharmaceutical formulations may include at least one of the disclosed peptides, compositions or pharmaceutically acceptable salt thereof in combination with a pharmaceutically acceptable carrier. Techniques of pharmaceutical production are well known in the art and typically include mixing a peptide, composition or salt in the presence of a suitable carrier. Suitable carriers for use with the peptides and compositions of the present invention include diluents, excipients, or carrier materials, selected according to the intended form of administration and consistent with conventional pharmaceutical or cosmetic practice. Examples of suitable carriers include, but are not limited to, water, physiological saline, phosphate-buffered saline, a physiologically compatible buffer, saline buffered with a physiologically compatible salt, a water-in-oil emulsion, and an oil-in-water emulsion, an alcohol, dimethylsulfoxide, dextrose, mannitol, lactose, glycerin, propylene glycol, polyethylene glycol, polyvinylpyrrolidone, lecithin, albumin, sodium glutamate, cysteine hydrochloride, and the like, and mixtures thereof. Suitable carriers can also include appropriate pharmaceutically acceptable antioxidants or reducing agents, preservatives, suspending agents, solubilizers, stabilizers, chelating agents, complexing agents, viscomodulators, disintegrating agents, binders, flavoring agents, coloring agents, odorants, opacifiers, wetting agents, pH buffering agents, and mixtures thereof, as is consistent with conventional pharmaceutical practice (“Remington: The Science and Practice of Pharmacy”, 20th edition, Gennaro (ed.) and Gennaro, Lippincott, Williams & Wilkins, 2000).

The peptides and compositions may be included in cosmetic formulations. Such inclusion may assist in the reduction or prevention of wrinkles or symptoms associated with aging. The peptides and compositions of the present invention may be adapted for use with cosmetic formulations according to the particular cosmetic.

The pharmaceutical and cosmetic formulations may be provided via a variety of routes. Suitable routes of administration may include oral, intestinal, parenteral, transmucosal, transdermal, intramuscular, subcutaneous, rectal, intramedullary, intrathecal, intravenous, intraventricular, intraatrial, intraaortal, intraarterial, or intraperitoneal administration. Though nonlimiting, it is believed the preferred in vivo route of administration will include topical application.

For use in a living, whole organism, such as in a human subject, compositions of the present invention can be formulated and provided in any formulation suitable to the intended form of administration and consistent with conventional pharmaceutical practice (“Remington: The Science and Practice of Pharmacy”, 20^{th} edition, Gennaro (ed.) and Gennaro, Lippincott, Williams & Wilkins, 2000). Examples of suitable formulations include tablets, capsules, syrups, elixirs, ointments, creams, lotions, sprays, aerosols, inhalants, solids, powders, particulates, gels, suppositories, concentrates, emulsions, liposomes, microspheres, dissolvable matrices, sterile solutions, suspensions, or injectables, and the like. Injectables can be prepared in conventional forms either as liquid solutions or suspensions, as concentrates or solid forms suitable for solution or suspension in liquid prior to injection, or as emulsions.

The present invention includes compositions that include two or more of the peptides provided herein. In other embodiments one, two or more of the peptides provide herein are provided together with a separate compound or therapy used to treat a condition associated with the skin such as wrinkles or decreased elasticity.

Nucleic acid sequences encoding the peptides of the present invention are also within the scope of the present invention. Accordingly, the present invention includes an isolated nucleic acid sequence encoding a peptide sequence according to any of SEQ ID NOS: 1-5 and 11-15. The nucleic acid sequence can be operably linked to eukaryotic or prokaryotic regulatory sequences including a promoter to allow for expression of a peptide mimetic of the present invention. Accordingly, vectors comprising DNA sequences encoding the peptides of the present invention, adapted for expression in a bacterial cell, a yeast cell, a mammalian cell such as a human fibroblast cell and other animal cells are also within the scope of the present invention. Expression vectors comprising the regulatory elements necessary for expression of the DNA in the bacterial, yeast, mammalian or animal cells are commercially available and can be used to operably link host cell expression regulatory sequences to the DNA sequence encoding the peptide.

The present invention also includes the recombinant prokaryotic or eukaryotic cells transformed with the nucleic acid sequences of the present invention. An example of a suitable host cells for use in accordance with the present invention include the prokaryotic organism E. coli and eukaryotic yeast cells such as those organisms selected from the genus Saccromyces. In addition, various mammalian cells may be utilized as hosts, including, for example, mouse fibroblast cells, human fibroblast cells, CHO cells, HeLa cells, etc. Expression vectors can be used to transfect the host cells by methods well known in the art such as calcium phosphate precipitation, DEAE-dextran, electroporation or micro injection.

### D. Methods

Methods of the present invention include methods of modulating the amount of at least one component of the extracellular matrix (ECM). Accordingly, methods of the present invention include those that maintain or repair the ECM. The methods may be used to treat patients having aged related skin conditions such as wrinkles, decreased elasticity and the like. The methods may be used to accelerate wound healing. The methods may be used as a preventative measure to retain a healthy skin appearance.

In some methods synthesis of at least one component of the extracellular matrix is increased in a fibroblast by contact the fibroblast with a peptide or composition the present invention, such as but not limited to at least one of the peptides provided in SEQ ID NOS: 1-5 or 11-15. In other embodiments, a peptide provided in any one of SEQ ID NOS: 1-5 is combined with any one of the peptides provided in SEQ ID NOS: 6-10 for contact. The methods of the present invention may be practiced in vitro or in vivo. If practiced in vitro, a biological sample such as a sample containing a fibroblast, preferably a human fibroblast, is contacted directly with a peptide or composition according to the present invention.

The peptides and compositions of the present invention may increase the synthesis of a variety of components that regulate or form part of the extracellular matrix. Among these include collagen such as collagens I, II and IV, elastin, fibronectin, thrombospondin, matrix metalloproteinase 1 (MMP1), matrix metalloproteinase 2 (MMP2), matrix metalloproteinase 7 (MMP7), and a tissue inhibitor of metalloproteinase (TIMP). The peptides and compositions of the present invention may increase the production of growth factors such as but not limited to connective tissue growth factor (CTGF), insulin-like growth factor-1 (IGF-1), hepatocyte growth factor (HGF), basic fibroblast growth factor (b-FGF) and vascular endothelial growth factor (VEGF). The peptides of the present invention may be used in to increase RNA levels or protein levels of the components and growth factors within the cell.

In a preferred method of the present invention, a peptide of the present invention or composition including the peptide, such as a pharmaceutical formulation or cosmetic formulation, is administered to an individual or subject in need thereof. Subjects may be treated with a therapeutically effective amount or dosage. A therapeutically effective dosage may vary somewhat depending on the peptide or composition, between patient to patient, and will depend on the condition of the patient and route of delivery. As general guidance, a dosage from about 0.1 to about 50 mg/kg may have therapeutic efficacy, while still higher dosages potentially being employed.

Additional nonlimiting features of the present invention are explained in greater detail in the following non-limiting examples.

### EXAMPLES

### Example 1

### Effect of CPP-Mimicking Peptides on Synthesis of Collagen I, Collagen II and Elastin in Dermal Fibroblasts In Vitro

The ability to interfere interaction of TAF4, BRG1 and N-CoR1 with the active transcriptional complex and stimulate synthesis of collagen I, collagen II and elastin was tested on 2 human dermal fibroblast cell lines obtained from elderly patients (81 and 79 years old). It has been shown that:
TAF4 suppression using siRNA stimulates expression of numerous genes in embryonic fibroblasts. (Mengus G, Fadloun A, Kobi D, Thibault C, Perletti L, Michel I, Davidson I. 2005. TAF4 inactivation in embryonic fibroblasts activates TGFbeta signaling and autocrine growth. EMBO J. 2005 Aug. 3; 24(15):2753-67.)
Brg1 inhibits numerous genes during development. (Inayoshi Y, Kaneoka H, Machida Y, Terajima M, Dohda T, Miyake K, Iijima S. 2005. Repression of GR-mediated expression of the tryptophan oxygenase gene by the SWI/SNF complex during liver development. J Biochem (Tokyo). 2005 October; 138(4):457-65)
N-CoR1 plays a crucial role in the repressive activity of diverse transcription factors (J. Frasor, J. M. Danes, C. C. Funk and B. S. Katzenellenbogen. 2005. Estrogen down-regulation of the corepressor N-CoR: Mechanism and implications for estrogen derepression of N-CoR-regulated genes. PNAS. vol. 102 no. 37, 13153-13157.)
SMAD7 functions as a component of negative feedback regulation that controls timining and extent of TGFbeta signaling (Zhu X, Topouzis S, Liang L F, Stotish RL. Myostatin signaling through Smad2, Smad3 and Smad4 is regulated by the inhibitory Smad7 by a negative feedback mechanism. Cytokine. 2004 Jun. 21; 26(6):262-72.)
TAF10 is one of the TATA box-binding protein-associated factors (TAFs), which constitute the TFIID complex.

Our own experimental data also show that TAF4, BRG1 and N-CoR1 are expressed at relatively high levels in aged fibroblasts whereas young fibroblasts have much lower expression of these factors.

Peptides
TAF4.1:

(SEQ ID NO: 11)

RPKKRKVRRRLATNSELVGTLTRSCKDETFLL
BRG1.1:

(SEQ ID NO: 12)

RPKKRKVRRRMGLGKTIQTIALITYLMEHKRINGP
N-CoR1.1:

(SEQ ID NO: 13)

RPKKRKVRRRSRWTEEEMEVAKKGLVEHGRNWAAIAKMVG
SMAD7.1:

(SEQ ID: 14)

RPKKRKVRRRGQLNSDHKSQLVEKVRLKIGCGIQL
TAF10.1:

(SEQ ID: 15)

RPKKRKVRRRNRAGFEASDPRIIRLISLAAQKFISDIANDALQ

### Methods

Human dermal fibroblasts were cultured in standard conditions (DMEM, 10% FCS, penicillin+streptomycin) and used in experiments after three passages in the laboratory. Cells were grown in 24 well plates, each treatment in triplicates. Cells were plated 16 hours prior treatments started. Peptides were added to the media, and media was changed every day during 7 day experiment. CPP concentration was 10 μM.

Western blot using antibodies against fibronectin, thrombospondin, collagen I, collagen II, collagen 4, elastin, MMP1, MMP2, MMP7 and TIMP1 were performed using standard protocols. Cells were lysed in SDS loading buffer and separated on 5% SDS PAGE following transfer onto PVD membrane using semidry blotting. Antibodies were diluted 1:2000 and incubated for 2 hours followed by secondary antibody conjugated to alkaline phosphatase. Antibody binding was visualized using one step BCIP reagent (Pierce).

### RNA Preparation

Total RNA from various human tissues was purified using RNAwiz (Ambion, USA) and subjected to subsequent DNase I treatment using the DNA-Free kit (Ambion). Total RNA from cancer cells was purified by using 4PCRmini kit (Ambion). First-strand cDNAs were synthesized with Superscript III reverse transcriptase (Invitrogen, USA) and 5 μg of total RNA using oligo d(T) priming in a final reaction volume of 50 μL

### RT-PCR Analysis

The cDNA was amplified by PCR using the 2,5U Hot-Firepol (Solis BioDyne, Estonia) and buffer Yellow (Naxo, Estonia). Reaction was performed in a 10 ul volume, using 0.5 ul cDNA as a template. Following primer sets were designed for this study:

TABLE 1
Effect of CPP mimicking peptides on synthesis of collagen and elastin

| | | | Fold increase after 7 days | | |
|---|---|---|---|---|---|
| Cells | Peptide | conc | collagen I | collagen II | elastin |
| fibroblast 1 | no peptide | | 1 | 1 | 1 |
| | TAF4.1 | 10 μM | 5 | 3 | 4 |
| | BRG1.1 | 10 μM | 7 | 2 | 3 |
| | N-CoR1.1 | 10 μM | 1.5 | 3 | 7 |
| fibroblast 1 | no peptide | | 1 | 1 | 1 |
| | TAF4.1 | 10 μM | 2 | 4 | 2 |
| | BRG1.1 | 10 μM | 3 | 5 | 6 |
| | N-CoR1.1 | 10 μM | 5 | 3 | 3 |

### Conclusion

Interfering peptides derived from TAF4, BRG1 and N-CoR1 stimulate expression of collagen I, collagen II and elastin in aged dermal fibroblasts in vitro.

TABLE 2
Effect of interfering peptides TAF10.1 and SMAD7.1 on
the level of expression of ECM components both at the
protein and mRNA level. Numbers show fold stimulation.

| | | | |
|---|---|---|---|
| Analyzed | control | peptide | peptide |
| protein, mRNA | peptide | TAF10.1 | SMAD7.1 |

PROTEINS

| | | | |
|---|---|---|---|
| Fibronectin | 1 | 2.6 | 3.1 |
| thrombospondin | 1 | 2.9 | 3.2 |
| CollagenI | 1 | 3.9 | 2.9 |
| collagen IV | 1 | 2.9 | 2.1 |
| Elastin | 1 | 2.4 | 2.6 |
| MMP1 | 1 | 1.3 | 1.9 |
| MMP2 | 1 | 1.5 | 1.1 |
| MMP7 | 1 | 1.7 | 1.9 |
| TIMP | 1 | 2.7 | 1.3 |

mRNA

| | | | |
|---|---|---|---|
| fibronectin | 1 | 9.8 | 8.7 |
| thrombospondin | 1 | 12.7 | 6.9 |
| collagenI | 1 | 7.9 | 7.9 |
| collagen IV | 1 | 9.0 | 5.4 |
| elastin | 1 | 4.7 | 7.7 |
| MMP1 | 1 | 3.9 | 3.6 |
| MMp2 | 1 | 7.9 | 1.9 |
| MMP7 | 1 | 1.9 | 2.9 |
| TIMP | 1 | 4.8 | 4.3 |

The cells were treated with peptides (1 μM) 24 hours for the mRNA analysis and 72 hours for protein analysis. All experiments were done in triplicates. Variation between replicates was less that 15%.

Presented data clearly demonstrate that peptides TAF10.1 and SMAD7.1 stimulate expression of components of ECM in dermal fibroblasts in vitro.

### Example 2

### Analysis of the Effect of Mimicking Peptides on the Activity of Elastin Promoter Using Transient CAT Assay

Numerous transcription factors interact and regulate elastin promoter (Lakkakorpi J, Li K, Decker S, Korkeela E, Piddington R, Abrams W, Bashir M, Uitto J, Rosenbloom J. 1999. Expression of the elastin promoter in novel tissue sites in transgenic mouse embryos. Connect Tissue Res. 1999; 40(2):155-162). Thus an elastin 5.2 kb reporter promoter construct was used to analyze effect of mimicking peptides on promoter activity using transient CAT assay.

### Method

Human dermal fibroblasts were cultured in standard conditions (DMEM, 10% FCS, penicillin+streptomycin) and used in experiments after three passages in the laboratory. Elastin 5.2 kb CAT promoter construct was used in all experiments.

Cells were transfected by using FuGene reagent (Roche Molecular Biochemicals) according to manufacturer's instructions. Freeze-thaw lysates of cells collected 48 h after the transfection were assayed for CAT activity as described (Pothier, F. et al., DNA Cell Biol. 11(1):83-90 (1992)). At least two different DNA preparations were tested for each plasmid. To normalize the transfection efficiencies, cells were cotransfected with pON260 expressing b-galactosidase (Spaete, R. R. and Mocarski, E. S., J. Virol. 54(3):817-24 (1985); Spaete, R. R. and Mocarski, E. S., J. Virol. 56(1):135-43 (1985)). All the CAT activities were normalized to total protein and b-galatosidase activity.

Peptides were as Follows:

(SEQ ID NO: 11)

| | |
|---|---|
| TAF4.1: | RPKKRKVRRRLATNSELVGTLTRSCKDETFLL |

(SEQ ID NO: 12)

| | |
|---|---|
| BRG1.1: | RPKKRKVRRRMGLGKTIQTIALITYLMEHKRINGP |

(SEQ ID NO: 13)

| | |
|---|---|
| N-CoR1.1: | RPKKRKVRRRSRWTEEEMEVAKKGLVEHGRNWAAIAKMVG |

Following transfection, cells were grown in 6 well plates, each treatment in triplicates. Peptides were added to the media and media was changed every day during the 5 day experiment. CPP concentration was 10 μM.

### Results

CAT assay data clearly show that in dermal fibroblasts isolated from elderly patients mimicking peptides stimulate promoter activity significantly whereas control suppress Dct/Trp2 promoter activity significantly whereas control peptides do not have a significant effect.

### Example 3

### Effect of Peptides N-CoR1.1, SMAD7.1 and TAF10.1 on Gene Expression Of Human Dermal Fibroblasts

### Peptides:

N-CoR1.1:

(SEQ ID NO: 13)

RPKKRKVRRRSRWTEEEMEVAKKGLVEHGRNWAAIAKMVG
SMAD7.1:

(SEQ ID NO: 14)

RPKKRKVRRRGQLNSDHKSQLVEKVRLKIGCGIQL
TAF10.1:

(SEQ. ID NO: 15)

RPKKRKVRRRNRAGFEASDPRIIRLISLAAQKFISDIANDALQ

### Methods

Human dermal fibroblasts were cultured in standard conditions (DMEM, 10% FCS, penicillin+streptomycin) and used in experiments after three passages in the laboratory. Cells were grown in 60 mm plates, each treatment in duplicates. Cells were plated 16 hours prior treatments started. Peptides (10 μM) were added to the media, and cells were collected for RNA isolation 24 hour later.

### RNA Preparation

Total RNA was purified by using 4PCRmini kit (Ambion). First-strand cDNAs were synthesized with Superscript III reverse transcriptase (Invitrogen, USA) and 5 μg of total RNA using oligo d(T) priming in a final reaction volume of 50 μL and used to generate fluorescently labeled probes for whole genome gene expression analysis.
Whole genome gene expression analysis was conducted using Affymetrix GeneChip™ Human Gene 1.0 ST Array.

### Results.

Whole genome gene expression analysis showed that peptides N-CoR1.1, SMAD7.1 and TAF10.1 affected expression of relatively large number of genes (Table 3) whereas both up- and down-regulation was observed.

TABLE 3
Effect of peptides N-CoR1.1, SMAD7.1 and TAF10.1on gene
expression in human dermal fibroblasts in vitro.

| | Number of genes with altered expression | | |
|---|---|---|---|
| Change | N-CoR1.1 | SMAD7.1 | TAF10.1 |

Expression stimulated

| | | | |
|---|---|---|---|
| More than 5 fold | 251 | 198 | 451 |
| 2-5 fold | 1192 | 986 | 786 |

Expression suppressed

| | | | |
|---|---|---|---|
| More than 5 fold | 76 | 124 | 298 |
| 2-5 fold | 1286 | 1011 | 543 |

### Example 4

### Effect of Peptides N-CoR1.1, SMAD7.1 and TAF10.1 on Expression of Growth Factors

Extracellular matix synthesized by dermal fibroblasts is also controlled by numerous growth factors and cytokines. Since whole genome gene expression analysis showed that expression of several growth factor genes is upregulated by the peptides N-CoR1.1, SMAD7.1 and TAF10.1 we analyzed effect of peptides on CTGF, IGF1, HGF, b-FGF and VEGF expression (Table 4).

### Peptides

TAF4.1:

(SEQ ID NO: 11)

RPKKRKVRRRLATNSELVGTLTRSCKDETFLL
BRG1.1:

(SEQ ID NO: 12)

RPKKRKVRRRMGLGKTIQTIALITYLMEHKRINGP
N-CoR1.1:

(SEQ ID NO: 13)

RPKKRKVRRRSRWTEEEMEVAKKGLVEHGRNWAAIAKMVG
SMAD7.1:

(SEQ ID: 14)

RPKKRKVRRRGQLNSDHKSQLVEKVRLKIGCGIQL
TAF10.1:

(SEQ ID: 15)

RPKKRKVRRRNRAGFEASDPRIIRLISLAAQKFISDIANDALQ

### Methods

Human dermal fibroblasts were cultured in standard conditions (DMEM, 10% FCS, penicillin+streptomycin) and used in experiments after three passages in the laboratory. Cells were grown in 12 well plates, each treatment in triplicates. Cells were plated 16 hours prior treatments started. Peptides were added to the media, and media was changed every day during 7 day experiment. CPP concentration was 10 μM.

Western blots using antibodies against growth factors were performed using standard protocols. Cells were lysed in SDS loading buffer and separated on 5% SDS PAGE following transfer onto PVD membrane using semidry blotting. Antibodies were diluted 1:2000 and incubated for 2 hours followed by secondary antibody conjugated to alkaline phosphatase. Antibody binding was visualized using one step BCIP reagent (Pierce).

### RNA Preparation

Total RNA from various human tissues was purified using RNAwiz (Ambion, USA) and subjected to subsequent DNase I treatment using the DNA-Free kit (Ambion). Total RNA from cancer cells was purified by using 4PCRmini kit (Ambion). First-strand cDNAs were synthesized with Superscript III reverse transcriptase (Invitrogen, USA) and 5 μg of total RNA using oligo d(T) priming in a final reaction volume of 50 μL

### RT-PCR Analysis

The cDNA was amplified by PCR using the 2,5U Hot-Firepol (Solis BioDyne, Estonia) and buffer Yellow (Naxo, Estonia). Reaction was performed in a 10 ul volume, using 0.5 ul cDNA as a template. Primer sets were designed to specifically amplify the nucleic acid sequence for the corresponding growth factor for this study:

TABLE 4
Effect of peptides N-CoR1.1, SMAD7.1 and TAF10.1 on expression
of growth factors (protein and mRNA). Numbers show the fold
stimulation of expression compared to control.

| | | | | |
|---|---|---|---|---|
| Growthfactors | control | N-CoR1 | SMAD7.1 | TAF10.1 |

mRNA

| | | | | |
|---|---|---|---|---|
| CTGF | 1 | 12 | 27 | 18 |
| IGF-1 | 1 | 2 | 4 | 2 |
| HGF | 1 | 5 | 8 | 2 |
| b-FGF | 1 | 23 | 2 | 8 |
| VEGF | 1 | 2 | 4 | 2 |

protein

| | | | | |
|---|---|---|---|---|
| CTGF | 1 | 9 | 15 | 13 |
| IGF-1 | 1 | 1.2 | 1 | 1 |
| HGF | 1 | 2 | 1.7 | 1 |
| b-FGF | 1 | 3 | 1 | 2 |
| VEGF | 1 | 1 | 1.3 | 1 |

All experiments were done in triplicates. Variation between replicates was less that 15%.
Presented results show that peptides N-CoR1.1, SMAD7.1 and TAF10.1 stimulate expression of CTGF significantly. Stimulation of CTGF that controls many aspects of ECM biology can contribute to the effect of peptides on the homeostasis of ECM.

What is claimed is:

1. A peptide capable of stimulating the synthesis of a component of an extracellular matrix (ECM) in a human fibroblast cell, wherein said peptide comprises an amino acid sequence selected from the group consisting of SEQ ID NO: 1, SEQ ID NO: 2, SEQ ID NO: 3, SEQ ID NO: 4 and SEQ ID NO: 5.

2. The peptide according to claim 1, wherein said peptide is less than or equal to 60 amino acid residues.

3. The peptide according to claim 1, wherein said peptide is less than or equal to 40 amino acid residues.

4. The peptide according to claim 1, wherein said peptide is less than or equal to 30 amino acid residues.

5. The peptide according to claim 1, wherein said peptide is a peptide mimetic.

6. The peptide according to claim 5, wherein said peptide mimetic is substantially similar to a domain selected from the group consisting of a transcription regulation domain, a transrepression domain and a protein:protein interaction domain of a transcriptional regulator.

7. The peptide according to claim 1, wherein said component is selected from the group consisting of a collagen, elastin, fibronectin, thrombospondin, matrix metalloproteinase 1 (MMP1), matrix metalloproteinase 2 (MMP2), matrix metalloproteinase 7 (MMP7), and a tissue inhibitor of metalloproteinase (TIMP).

8. The peptide according to claim 7, wherein said collagen is selected from the group consisting of collagen I, collagen II and collagen IV.

9. The peptide according to claim 1, wherein said peptide comprises an amino acid sequence selected from the group consisting of SEQ ID NO: 11, SEQ ID NO: 12, SEQ ID NO: 13, SEQ ID NO: 14 and SEQ ID NO: 15.

10. The peptide according to claim 9, wherein said component is selected from the group consisting of a collagen, elastin, fibronectin, thrombospondin, matrix metalloproteinase 1 (MMP1), matrix metalloproteinase 2 (MMP2), matrix metalloproteinase 7 (MMP7), and a tissue inhibitor of metalloproteinase (TIMP).

11. The peptide according to claim 1, wherein said peptide further comprises an amino acid sequence selected from the group consisting of SEQ ID NO: 6, SEQ ID NO: 7, SEQ ID NO: 8, SEQ ID NO: 9 and SEQ ID NO: 10.

12. A composition comprising at least one peptide according to claim 1.

13. A pharmaceutical composition comprising the peptide of claim 1, and a pharmaceutically acceptable carrier or a physiologically acceptable carrier.

14. A method of preventing or treating a skin condition associated with aging, comprising administering the pharmaceutical according to claim 13 to an individual suffering from or at risk of developing the skin condition.

15. A method of stimulating the synthesis of a component of an extracellular matrix (ECM) in a cell, comprising providing a human fibroblast cell and administering to said fibroblast cell a peptide according to claim 1 in an amount sufficient to stimulate synthesis of the component.

16. The method according to claim 15, wherein said component is a collagen or elastin.

17. A method of stimulating the synthesis of a component of the extracellular matrix, comprising providing a human dermal fibroblast and administering to said cell a peptide according to claim 9.

18. A method of increasing the presence of RNA corresponding to a component of the extracellular matrix in a human fibroblast cell comprising providing the human fibroblast cell and contacting said human fibroblast with a peptide according to claim 9 in an amount sufficient to increase the presence of the RNA.

19. A method of increasing synthesis or presence of a component of the extracellular matrix (ECM) in a cell having a dysfunctional transcription factor selected from the group consisting of TAF4, SMAD7, E-CoR1 and Taf10, the method comprising providing a cell having the dysfunctional transcription factor and contacting the cell with a peptide according to claim 9.
